(19) United States
(12) Patent Application Publication
(10) Pub. No.: US 2023/0200637 A1
(43) Pub. Date: Jun. 29, 2023

(54) DEVICES AND METHODS FOR ENDOSCOPIC NEUROABLATION IN THE TYMPANIC CAVITY

(71) Applicants: Dorna HAKIMIMEHR, Redwood City, CA (US); Suresh Subraya PAI, Los Altos, CA (US); Marc Richard DEAN, Dallas, TX (US)

(72) Inventors: Dorna HAKIMIMEHR, Redwood City, CA (US); Suresh Subraya PAI, Los Altos, CA (US); Marc Richard DEAN, Dallas, TX (US)

(21) Appl. No.: 18/068,350

(22) Filed: Dec. 19, 2022

Related U.S. Application Data
(60) Provisional application No. 63/294,760, filed on Dec. 29, 2021.

(51) Int. Cl. A61B 1/227; A61B 18/14; A61B 18/02; A61B 1/05; A61B 1/06

(52) U.S. Cl. CPC A61B 1/227; A61B 18/1482; A61B 18/02; A61B 1/05; A61B 1/0676; A61B 2018/00327

(57) ABSTRACT

The various embodiments described herein provide devices and methods for neuroablation in the tympanic cavity. Devices include an ablation effector that can be navigated into the tympanic cavity to ablate nerves therein in order to treat diseases caused due to the malfunction of these nerves.

### CROSS-REFERENCE TO RELATED APPLICATIONS

This application claims the benefit of priority to U.S. Prov. App. 63/294,760 filed Dec. 29, 2021, which is incorporated herein by reference in its entirety.

### BACKGROUND OF THE INVENTION

The middle ear cavity, also known as the tympanic cavity, is an air-filled chamber in the petrous part of the temporal bone. It is separated from the external ear by the tympanic membrane, and from the inner ear by the medial wall of the tympanic cavity. It contains the three auditory ossicles whose purpose is to transmit and amplify sound vibrations from the tympanic membrane to the oval window of the lateral wall of the inner ear.

Middle ear ossicles consist of three small bones (the malleus, incus and stapes), which form a mobile chain across the tympanic cavity from the tympanic membrane to the oval window. There are two muscles, one attached to the malleus, and one attached to the stapes, which act to damp down over-vibration from low-pitched sound waves. The tensor tympani muscle inserts into the handle of the malleus, and stapedius muscle inserts into the neck of the stapes.

### A. Middle Ear Innervation

### A1. Chorda Tympani

The chorda tympani, a branch of nervus intermedius, leaves the facial nerve in the facial canal and enters the tympanic cavity through the posterior wall, lying just underneath the mucous membrane. It runs over the pars flaccida of the tympanic membrane, and the neck of the malleus. It leaves at the anterior margin of the tympanic notch. The chorda tympani carries special visceral afferent fibers that relay sensations of taste from the anterior portion of the tongue. Additionally, it carries parasympathetic general visceral efferent fibers to the submandibular ganglion, which then innervate the sublingual and submandibular glands. The chorda tympani's efferent parasympathetic fibers cause vasodilation of blood vessels in the tongue.

Damage to the chorda tympani nerve can occur due to inflammatory diseases of the middle ear such as otitis media and cholesteatoma. It can also result from medication use, radiotherapy, viral infection, Bell's Palsy, or due to surgical intervention in the middle ear. Damage to the chorda tympani, regardless of cause, can result in impaired secretion of the lacrimal, submandibular, and sublingual glands, and decreased taste sensitivity or distortions in taste perception. In other conditions, for example in case of excessive drooling, the reduced function of the chorda tympani might be desirable. Pathologies associated with the function of chorda tympani include, but are not limited to phantom taste, retronasalretroasal olfaction, obesity (hypoactice chorda tympani), and burning mouth syndrome.

### A2. Jacobson's Nerve

The tympanic branch of the glossopharyngeal nerve (Jacobson's nerve) forms the tympanic plexus by combining with sympathetic fibers from the internal carotid plexus (caroticotympanic nerves) and a small communicating branch with the geniculate ganglion of the facial nerve. The caroticotympanic nerves are nerves which supply the eardrum (“tympanum”) and carotid canal. They are the postganglionic sympathetic fibers from internal carotid plexus which enter the tympanic cavity via the caroticotympanic artery. The tympanic plexus gives off the lesser petrosal nerve (LPN), which provides parasympathetic innervation to the parotid gland via the otic ganglion. The LPN originates at the region of the geniculate ganglion and carries mainly preganglionic parasympathetic fibers from the tympanic plexus to the parotid gland. Although its largest source of fibers is from the tympanic branch of the IX, the LPN is actually the result of a composition of fibers contributed by three different nerves: 1) the tympanic branch of the IX, 2) the nervus intermedius of the VII, and 3) the auricular branch of the X (Arnold's nerve) (Mavridis & Pyrgelis, 2016).

The preganglionic parasympathetic fibers synapse with the cell bodies of the postganglionic parasympathetic fibers in the otic ganglion (OTG). The sympathetic fibers that join the nerve from the middle meningeal artery pass right through the OTG. The postganglionic parasympathetic secretomotor fibers from the OTG pass to the parotid salivary gland through the auriculotemporal nerve which lies in contact with the deep surface of the gland (Mavridis & Pyrgelis, 2016).

### A3. Arnold's Nerve

The auricular branch of the vagus nerve (Arnold's nerve) conveys sensation from the tragus and external acoustic meatus. It is a mixed general somatic afferent nerve composed of vagal, glossopharyngeal, and facial nerve fibers. Arnold's nerve is the remnant of the embryonic nerve that supplies the first branchial arch, which includes the external acoustic meatus, middle ear and auditory tube. This nerve enters the temporal bone on the external surface of the jugular fossa and runs horizontally where a small branch unites it with the facial nerve. Turning around the posterior aspect of the facial nerve, it comes to be immediately behind the chorda tympani to which it connects then turns more abruptly downwards and leaves the bone through a small foramen close to the stylomastoid foramen. Laryngeal cancer and myocardial infarction can both present with pain behind the ear and in the ear—this is a referred pain through the vagus nerve to Arnold's nerve.

### A4. Facial Nerve

The labyrinthine segment of facial nerve begins after the facial nerve has passed through the internal acoustic meatus. After the internal acoustic meatus, the motor root of the facial nerve, and the nervus intermedius enter the facial canal, and they then pass between the cochlea and vestibule before bending posteriorly at the geniculate ganglion, at which point the motor root and nervus intermedius join. The labyrinthine segment gives off three branches including the greater superficial petrosal nerve (containing parasympathetic fibers for the lacrimal gland and taste fibers from the palate. The tympanic segment of the facial nerve (VII) begins as the facial nerve passes posteriorly at the geniculate ganglion. This segment is in the medial wall of the middle ear cavity, directly below the lateral semicircular canal. The mastoid segment begins after the tympanic segment moves downwards, distal from the pyramidal eminence of the middle ear cavity. From here, the mastoid segment travels through the facial canal and up to the stylomastoid foramen. This segment gives off three branches: the nerve to stapedius muscle, the chorda tympani (containing parasympathetic fibers to sublingual and submandibular salivary glands and taste fibers from the anterior two-thirds of the tongue), and the sensory branch which joins the auricular branch of the vagus nerve. The sensory branch carries general somatic afferent fibers from the pinna and the external auditory meatus (Seneviratne & Patel, 2022).

### B. Middle Ear Neural Pathologies

### B1. Otitis Media

Otitis media is a group of inflammatory diseases of the middle ear. One of the two main types is acute otitis media, an infection of rapid onset that usually presents with ear pain. The other main type is otitis media with effusion which is defined as the presence of non-infectious fluid in the middle ear which may persist for weeks or months. Otitis media with effusion is typically not associated with symptoms, although occasionally a feeling of fullness is described. Chronic suppurative otitis media is middle ear inflammation that results in a perforated tympanic membrane with discharge from the ear for more than six weeks.

Both immune and peripheral nervous systems are involved in both the acute and chronic phases of otitis media. The middle ear has an abundance of immune cells (such as mast cells), sensory neurons, and vasculature, which collectively participate in the immunological response of the ear to environmental stimuli. Upon stimulation or pathogen encounter, the sensory neurons release neuropeptides, mostly substance P and cGPRC to activate the immune system. In response to the neuropeptide release, mast cells release cytokines (such as TNF beta) as part of a defense mechanism. The neuropeptides then act on cytokines to induce positive feedback of inflammation.

It has been shown in animal and in vitro models that selectively ablating sensory neurons is effective in preventing formation of middle ear effusion or to treat ear hypersensitivity. Such selective ablation has also been shown to have positive effect on preventing bone remodeling due to middle ear infection and inflammation. This research is in its infancy and methods for causing such selective neurulation in humans and its potential efficacy have not been studied or contemplated.

### B2. Otalgia

The internal, middle and external ear are also supplied by branches of the trigeminal, facial, glossopharyngeal and vagus nerves which can result in referred pain in the ear from other areas supplied by these nerves, e.g., the teeth, the posterior part of the tongue, the pharynx, the larynx, or the temporomandibular joint.

Otalgia or earache is a common and indiscriminate affliction, affecting persons of all ages. When the cause of otalgia can be identified to be localized to the affected ear, it is referred to as a primary otalgia. Common causes of primary otalgia include otitis media, external otitis, folliculitis, cerumen impaction, mastoiditis, myringitis, and neoplasm. When the source of the pain cannot be localized to the affected ear, it is referred to as secondary or referred otalgia. Because of the complex interplay of multiple upper cervical, lower cranial, and peripheral nerves providing sensory information to and from the ear, localizing the source of referred ear pain is challenging. This complexity is magnified by the diverse anatomic territory innervated by these nerves, which spans portions of the brain, spine, skull base, aerodigestive tract, salivary glands, paranasal sinuses, face, orbits, deep spaces of the neck, skin, and viscera.

Any irritative focus such as tumor, infection, or inflammation of structures within the oral cavity (specifically including the floor of mouth, cheek, anterior tongue, hard palate, and sublingual and submandibular glands), lower teeth, mandible including the temporomandibular joint (TMJ), and parotid glands can all be sites of distant pathology resulting in referred otalgia. Dental diseases account for most pathology causing referred otalgia. Otalgia is listed as a chief complaint by 70%-78% of patients with TMJ disorders. It is unclear whether this is a true reflex referred ear pain secondary to direct impingement of the auriculotemporal nerve related to masseteric muscle spasm, or secondary to a direct ligamentous connection between the TMJ and middle ear, but one or all of these theories may potentially explain TMJ pathology resulting in otalgia (Chen et al., 2009).

Essentially any pathology residing within the sensory net of cranial nerves V, VII, IX, and X and upper cervical nerves C2 and C3 can potentially cause referred otalgia (Chen et al., 2009).

Division of Jacobson's nerve, or tympanic neurectomy, has been used as a successful treatment for referred otalgia. Tympanic neurectomy was first proposed by Lempert in 1946 for the treatment of tinnitus and was named “Tympanosympathectomy” (LEMPER, T, 1946). Because this procedure failed to relieve tinnitus, it was not further considered. In 1952, Rosen elicited the pain in two patients by stimulating Jacobson's nerve with an electrode (Rosen, 1952). Resection of the tympanic plexus in both patients led to complete resolution of their symptoms after which an additional series supported the success of this technique (Rosen, 1952).

The procedure was then described by Golding-Wood in 1962 and again repeated in 1990 by Cook et al. (Cook et al., 1990; Golding-Wood, 1962). In these published cases, referred otalgia was relieved following a tympanotomy with 2 mm of the tympanic nerve excised (Cook et al., 1990). In 2016, the procedure was revisited by Roberts et. al. who performed a microscopic tympanic neurectomy on twelve patients showing improvement in intractable otalgia (Roberts et al., 2016).

In the reported procedures, an operative microscope was used to raise a tympanomeatal flap. A section of Jacobson's nerve was then surgically removed. The tympanomeatal flap was returned and sutured to the posterior canal, and packing was placed in the external ear canal.

### B3. Headache

Nerve projections from the superior cervical ganglion (SCG), dorsal root ganglia (DRG) and otic ganglion (OTG) onto cerebral arteries have shown to contain neurotransmitters that can participate in migraine pathology. The effects of these ganglia have been investigated less intensively compared to that of the trigeminal ganglion and sphenopalatine ganglion (SPG) with respect to primary headaches (White et al., 2021). In addition to the ophthalmic branch of the trigeminal nerve, the maxillary (V2) and mandibular (V3) branches of the trigeminal nerve also participate in sensory innervation of the cerebral arteries and middle cranial fossa. Meningeal branches of V2 and V3 run along with branches of the middle meningeal artery (MMA). These intracranial branches also receive afferents from peri-cranial temporal, parietal, and occipital periosteum, and adjacent deep layers of the temporal and upper neck muscles, that could result in tension type headaches upon stimulation (Uebner et al., 2014). While V2 and V3 provide sensory innervation to MMA, superior cervical ganglion provides sympathetic innervation to this artery. Meanwhile, SPG and otic ganglion (OTG) provide MMA with parasympathetic innervation (Edvinsson et al., 1989; Uddman et al., 1989)

The sensitization of sensory neurons innervating MMA can result in a parasympathetic reflex arc, resulting in release of parasympathetic neurotransmitters such as VIP and PCAP from SPG and OTG which results in further vasodilation and prolongation of pain. The sympathetic/parasympathetic imbalance could similarly result in cerebral vasodilation which can lead to headache and migraine.

The parasympathethic feed into the OTG is formed within the tympanic cavity where branches of tympanic branch of the glossopharyngeal nerve mix with the sympathetic branches from the internal carotid artery and branches from the facial nerve to form a plexus. The plexus sends preganglionic parasympathetic fibers which synapse in the OTG to form post-synaptic parasympathetic fibers which then innervate MMA in addition to providing parasympathetic innervation to all branches of V3. While blocking SPG has been explored in treating migraine, it is reasonable to assume that blocking OTG or its parasympathetic fibers could potentially provide pain relief at least for a subset of migraine patients.

### B4. Neurogenic Inflammation

Inflammatory diseases in temporomandibular joint such as arthritis have been shown to results in increase in CGRP content in the trigeminal ganglia and the increased vasoactive intestinal peptide (VIP) accumulation in the Otic ganglion (Carleson et al., 1997). In case of inflammatory diseases of neurogenic origin disconnecting the parasympathetic outflow to the inflamed organ reduces vasodilation and can therefore terminate the inflammatory cascade. Similar strategy has been used in treatment of inflammatory diseases of the upper airways where cutting the parasympathetic outflow has resulted in relief from symptoms e.g. in case of ablation of posterior nasal nerve in treating vasomotor rhinitis or in treating migranious headache. Therefore, it is hypothesized that ablation of tympanic plexus with special interest in depleting its parasympathetic content could provide a potential treatment option.

B5. Obesity

The relationship between taste and obesity is complex. The ability to detect a tastant is hypothesized to be weakened by obesity and obese individuals report taste hyposensitivity. One potential candidate for decreased orosensory fat perception is the innervation of lingual fat receptors. Two nerves carry information from papillae to the central nervous system for further processing—the chorda tympani (CT) and the glossopharyngeal (GL). The CT primarily innervates the fungiform papillae and the anterior foliate papillae. The GL innervates the posterior foliate and the circumvallate papillae. In the clinical population, damage to either the CT, from repeated episodes of otitis media, or GL damage from head and neck pathology, lead to decreased taste sensation and these individuals were more likely to have obesity (Schreiber et al., 2020).

The “oral disinhibition” or “release of inhibition” model has been put forward, in which the sensory nerves of the mouth partially and mutually suppress one another, so that damage to one nerve disinhibits those that remain, resulting in increased activity that effectively compensates for regional loss. In human experiments, CT anesthesia enhances taste cues on the contralateral posterior tongue (innervated by IX). Other observations indicate that glossopharyngeal nerve loss drives CT disinhibition in a similar manner, and that damage to either nerve leads to elevated somatosensory input from the trigeminal nerve (Snyder & Bartoshuk, 2016).

In a recent study Schriber et al have shown that hyposensitive and/or dysregulated orosensory perception of highly palatable foods contribute to the susceptibility to develop obesity and that simultaneous sectioning of both CT and GL can overcome the compensatory effect on each other and effectively reduce fat intake in obesity prone rats (Schreiber et al., 2020).

### B6. Temporal Bone Hyper Pneumatization

Hyper pneumatization of the temporal bone or mastoid cells within the ear cavity can results in exposure of nerves that are normally encased in bony canals such as facial nerve. Symptoms observed in these cases depend on the exposed nerves and their field of innervation.

### Procedure

An operating microscope is typically used when performing middle ear surgery because of its ability to illuminate and magnify the surgical field. However, these microscopes only provide a line-of-sight image with a narrow field of view that is limited by the size and shape of the external auditory canal. The use of these microscopes also require elevation of the tympanic membrane to access and facilitate line of sight visualization of the middle ear structures. Endoscopes overcome many of these limitations providing a non-line-of-sight view that is less limited by port size and shape, with both illumination and image capture taking place at the distal tip of the scope near the surgical field.

Neurectomy, neurolysis, or destruction of neural tissue, can be performed surgically under direct visualization as conducted in the previously described tympanic neurectomy cases. As advancements in imaging and ablative techniques continue to progress, these procedures can now be done chemically, thermally via heating (e.g., application of radiofrequency, microwave, or laser energy) or via freezing (e.g. using cryo-neurolysis). Using these ablative techniques especially in combination with an imaging modality allows these complex procedures to be performed on a precise target without the need for extensive or more invasive surgery to create surgical access to the targeted middle ear anatomy.

There is limited prior work in using freezing or cryogenics for neurolysis and/or ablation in the tympanic cavity. House et al. investigated applying cryosurgery to the promontory or the horizontal canal of the tympanic cavity to selectively destroy the vestibular portion of the labyrinth by creating a perilymph-endolymph shunt or fistula to treat Meniere's disease (House, 1966). However, this technique did not result in any significant advantage over other known treatment modalities. Consistent with observations made by House, Bernstein and Friedmann saw little histological change in the labyrinth of a squirrel monkey after cryo application to the promontory (Bernstein & Friedmann, 1969). Due to lack of clinical efficacy, this specific application of cryosurgery in the tympanic cavity was not pursued further. To clarify, the ablation procedure discussed by these authors was intended to ablate the labyrinth through ossicular bone and was not intended to ablate a nerve within the tympanic cavity.

The current invention describes an ablation device and its minimally invasive method of use to perform targeted neural ablation procedures in the middle ear under direct endoscopic visualization. It also teaches devices and methods for transtympanic nerve ablation within the middle ear cavity without raising a tympanic flap and therefore leaving the tympanic membrane largely intact and minimizing trauma to other nerves in the middle ear cavity.

It is contemplated that the devices and methods of the invention described herein will enable practitioners to manage or treat several diseases and dysfunctions caused by the malfunctioning of the nerves passing through the tympanic cavity including secondary otalgia and taste disturbances. These outcomes can be achieved by temporary, minimally invasive neuroablation of middle ear nerves under direct visualization. It is contemplated that the temporary neural damage caused by the ablation will “disconnect” a misfiring nerve prompting neural regeneration. The newly regenerated nerve would lack the dysfunction and therefore resolve the affliction.

Use of the methods and devices described herein allow for surgical operations on middle ear nerves without damaging other middle ear structures, reducing the risk of these procedures. Additionally, use of energy-based ablation modalities such as heat or freezing allow for a larger section of the nerve to be ablated compared to single point nerve dissection, increasing the efficiency of the procedure, as non-ablated remnants of a nerve can compensate for the injured section and continue to transmit pain or send aberrant signals rendering the procedure less effective.

### SUMMARY OF THE INVENTION

In one embodiment, the device comprises a proximal end and distal end connected by an elongate shaft with a central lumen. The proximal end is to be held and manipulated by an operator (e.g., a surgeon) to navigate the distal end to the tympanic cavity of a person via the external ear canal and through the tympanic membrane. The distal end of the device is intended to effect ablation and as such comprises an ablation effector component.

In another embodiment, in addition to the ablation effector, the distal end of the device may also include an image capture component (e.g., a miniature or subminiature CMOS imaging sensor), and/or an illumination component (e.g., a plastic or glass fiber in communication with a light source or a light emitting diode or LED) facilitating direct visualization of the middle ear anatomy. The image captured by the image capture component (e.g., the component's output) is displayed on a monitor or other display device (e.g., smartphone or tablet) via an image processing component that is either housed within the proximal end, physically connected through a communication cable or the like to the proximal end, or is alternatively in wireless communication (e.g., using Bluetooth or WIFI) with the proximal end of the device. The proximal end may also be used to manipulate and control the device during the ablation procedure.

In another embodiment, in addition to the ablation effector, image capture and illumination components, the distal end comprises a working channel to advance surgical tools such as an otology (ear) pick to the surgical site, allowing additional operations to be conducted under endoscopic visualization. The proximal end of said working channel can be optionally attached to a vacuum source to aspirate excess fluids and the like from the surgical field to improve visualization.

In another embodiment, in addition to the ablation effector, image capture and illumination components, the distal end includes and incorporates a cutting component that can be used to create an incision in the tympanic membrane providing access to the tympanic cavity.

In one embodiment of the device where the device includes image capture and/or illumination components, the ablation effector is arranged so it can slide axially in relation to the other components of the distal end. For example, in one configuration, the ablation component is arranged in parallel with the image capture and the illumination components during the navigation of the distal end to the target site. Once the device reaches the proximity of the target tissue, the ablation component is slid forward (e.g., distally towards the promontory in the middle ear space) in relation to the other components of the distal end, placing the ablation effector in the field of view (i.e., in front of) the image capture component allowing the operator to monitor, observe and control the ablation procedure.

In one example, the mechanism to control the axial sliding movement of the ablation effector is housed within the proximal end of the device. Therefore, the operator can control the movements of the ablation component from the proximal end of the device. In another example, the mechanism to control the axial sliding movement of the ablation effector is housed within an external structure where the external structure is in electrical and/or wireless communication with the proximal end of the device.

In one embodiment, the device's entire elongate shaft is substantially straight. In another embodiment, a portion of the distal end of the elongate shaft is bent at an angle of about 0-120 degrees in relation to a portion of the elongate shaft. Positioning the distal end and therefore the ablation effector at an angle to a portion of the elongate shaft can facilitate ablation of a greater surface area of target tissue or provide access to tissues that are not directly inline with the entry point at the tympanic membrane. In one example, the distal portion of elongate shaft can be prefabricated to have a bend or angle in relation to the rest of the elongate shaft or alternatively the distal portion of the elongate shaft may include a malleable or bendable portion and/or connection that can be manipulated to the desired bend angle by the operator. In yet another embodiment, the connection between the distal end and the shaft may be rendered steerable (using methods commonly known in the art) to allow the operator to intra-procedurally control the bend angle of distal end of the elongate shaft relative to the rest of the elongate shaft as needed.

In one embodiment of the device, some or all components of the distal end of the device are designed to have a collapsed and an expanded configuration. During navigation of the distal end of the device through the external ear canal to the tympanic cavity, parts or all of the distal end may be in a collapsed state facilitating navigation. Once inside the tympanic cavity, part or all of the distal end can be expanded to increase the contact area between the distal end and the target tissue. Once the ablation procedure is completed, all or part of the distal end that has been expanded can return to the collapsed state to ease and facilitate atraumatic navigation of the device out of the tympanic cavity. In one example of this embodiment, the ablation effector is the only component that can change from a collapsed to an expanded state.

In another embodiment where the distal end includes image capture and/or illumination components, once the distal end of the device reaches the tympanic cavity, the ablation component is slid axially forward in relation to other components of the distal end and is transformed from a collapsed state to an expanded state. Upon the completion of the procedure, the ablation component is collapsed, slid backward to be inline with other components of the distal end, and the distal end is navigated out of the tympanic cavity.

In one embodiment of the device, the ablation effector is a cryosurgical device or a cryoprobe operating under the Joules-Thompson effect to cause extreme cold temperatures capable of effecting nerve ablation. In this example, the cryosurgical probe or cryoprobe is provisioned with a gas conduit for delivering pressurized cryogenic gas or liquid from a gas or liquid source to the probe head and another, preferably coaxial, conduit for removing the excess coolant gas away from the probe head. The cryogenic material may be stored in a cartridge, capsule or other suitable container, sized to be housed within the proximal end of the device or separately from the device with the gas or liquid ported to the device via a tube in fluid communication with the cryoprobe lumen. The operator can control the ablation procedure either using a switch or other control mechanism on or within the proximal end or alternatively using a foot pedal or other means of controlling the ablation, located away from the proximal end.

In another embodiment where the distal end includes image capture and/or illumination components, the cryoprobe may be insulated to be thermally isolated from the rest of the components of the device to prevent or reduce the potential for thermal damage to such components and to limit or prevent collateral damage to non-target tissues (e.g. the tympanic membrane). In yet another embodiment, a heating element can be incorporated within the device at the proximal end, distal end, and/or along the elongate member to maintain the temperature of the image capture and/or illumination components at desirable operating temperatures. Said heating element may also be beneficial in preventing or reducing collateral damage to non-target tissues (e.g. the tympanic membrane or to walls of the external ear canal) by modulating or reducing cold temperatures by any cryogenic material flowing within areas of the device or along the elongate member. The heating elements in this embodiment may comprise thermofoil heaters and flex circuits. The cryoprobe may optionally be slidably disposed in relation to other components of the distal end allowing it to be extended towards the target tissue once the device has passed through the incision in the tympanic membrane.

In one example of this embodiment, the cryoprobe operates on the basis of the Joules-Thomson effect and is in form of an expandable balloon. Once the probe is in proximity of target tissue, the coolant gas or liquid used to cause extreme cold temperatures can be used to expand the balloon. Upon the completion of the procedure, the flow of the cryogenic gas or liquid is stopped resulting in the balloon deflating to substantially its original size and returning to environmental temperature. The cryoprobe (balloon) can then be navigated to a subsequent ablation target in the middle ear or alternatively retracted out of the tympanic cavity.

In one embodiment, the ablation effector is a cryoprobe that uses nitrous oxide liquid/gas to produce the cold temperature. In another embodiment, the cryoprobe uses carbon dioxide. In yet another embodiment the cryoprobe uses liquid nitrogen. In one embodiment, the cryoprobe uses chlorofluorocarbon, hydrochlorofluorocarbon, hydrofluorocarbon or any mixtures thereof as cryogenic material.

In another embodiment, the cryoprobe uses principles other than the Joules-Thompson effect to produce cold (cryogenic) temperature conditions.

In another embodiment of the device, the ablation effector is a cryoprobe that sprays cryogenic material directly onto the target tissue in the middle ear space to effect ablation.

In one embodiment of the device of the current invention, the ablation effector uses heat to effect ablation. Different energy modalities such as bipolar, radiofrequency, pulsed radiofrequency, ultrasonic and microwave can be used to heat the target tissue and effect ablation.

In one embodiment, the ablation component delivers liquid or semi-liquid chemicals onto the target tissue to effect ablation. Examples of such chemicals include, but are not limited to ethanol, phenol, formulations or combinations thereof.

In another embodiment, the ablation component delivers liquid or semi-liquid therapeutic agents to induce ablation. Examples of such agents includes, but are not limited to capsaicin and its analogues (e.g. Zucapsaicin, ALGRX-4975, Nonivamide, Resiniferatoxin, or combinations thereof), or sympatholytic agents such as alpha- and beta-adrenergic receptor antagonists (e.g., alpha and beta blockers), as well as centrally acting agents such as clonidine, guanabenz, methyldopa, minoxidil, and reserpine.

Methods of using the devices described may include using the devices to ablate a nerve within the tympanic cavity to treat diseases related to dysfunction of the said nerve. According to one method, the surgeon consults with the patient to ascertain that the patient is a candidate for the current procedure. After applying appropriate anesthesia, the surgeon uses a surgical tool or alternatively the devices described herein to create an incision in the tympanic membrane of the patient. The distal end of the device is then navigated through the external ear canal under direct visualization to enter the tympanic cavity through the incision in the tympanic membrane. The visualization component of the device is used to safely navigate the anatomy and to visually identify the target nerve or nerves once positioned within the tympanic cavity. The ablation effector of the distal end of the device is positioned at the desired location on the nerve trajectory followed by ablation of the said nerve or nerves. For this step, the ablation effector is positioned in direct contact with all or a portion of the nerve or nerves targeted for ablation under direct endoscopic visualization. Once the desired amount of the target nerve or nerves is/are ablated, the ablation procedure is stopped. It is envisioned that the ablation procedure may be optionally repeated multiple times at the same or different locations along the nerve or its branches to achieve the desired therapeutic effect. Once the ablation is completed, the device is navigated (e.g., retracted) out of the tympanic cavity through the incision in the tympanic membrane and out of the external ear canal under direct visualization. Post operatively, the external ear canal may be packed with appropriate packing material to accelerate healing of the incision in the tympanic membrane.

In an alternative method of using the device, in lieu of creating an incision in the tympanic membrane to gain access to the tympanic cavity, the surgeon uses a surgical tool to create an incision in the external ear canal and elevates a portion of the tympanic membrane in the form of a tympanomeatal flap. The distal end of the device is then navigated through the external ear canal under direct visualization to enter the tympanic cavity to perform the ablation procedure. Upon completion of ablation, the device is navigated out of the tympanic cavity and external ear canal. The tympanomeatal flap is then retuned and secured, sutured or otherwise attached to its original position as surgically practicable. In another embodiment facial recess procedure is used to access tympanic cavity.

In yet another method of using the device, the ablation device does not include or incorporate visualization components (e.g., image capture and/or illumination components). In this instance, after confirming the patient to be a candidate for the procedure and after applying appropriate anesthesia, the surgeon uses a surgical tool to either create an incision in the tympanic membrane or alternatively create an incision in the patient's external ear canal and raise a portion of the tympanic membrane in form of a flap or use a facial recess procedure. A visualization tool e.g. a traditional rigid or flexible endoscope or a microscope is used to perform the procedure. The distal end of the device is then navigated through the external ear canal adjacent to said visualization tool to enter the tympanic cavity under direct visualization. Upon completion of the ablation procedure the treatment device and the visualization tool are navigated out of the patient's ear either in sequence or together.

In yet another method for treating dysfunctions or diseases in a patient, the method may generally comprise creating access to a tympanic cavity of a patient, introducing a treatment device into the tympanic cavity of the patient, the treatment device having a proximal end, a distal end, an elongate shaft therebetween, and an ablation effector disposed at or near the distal end of the device, advancing the distal end of the device under direct visualization into proximity of a nerve or nerves within the tympanic cavity, and ablating the nerve or nerves using the ablation effector to reduce at least one symptom of nerve dysfunction or disease.

### BRIEF DESCRIPTION OF THE DRAWINGS

FIG. 1A shows the schematic side view of the structures of an ear.

FIG. 1B is the schematic of structures, including the nerves, within the tympanic cavity (middle ear) of an ear.

FIG. 2A shows the schematic of the device of the current invention accessing the tympanic cavity through the external ear canal and tympanic membrane.

FIG. 2B shows the distal end of the device of the current invention advanced through the incision in the tympanic membrane and positioned within the tympanic cavity in proximity with or in direct contact with some or all of the nerve(s) targeted for ablation.

FIG. 3A shows a schematic cross-sectional view of the distal end segment of the device of the invention along with its components.

FIG. 3B-3D shows schematics of various configurations of the ablation effector component of the distal end segment of the device in relation to other components of the device.

FIGS. 4A-4C shows various configurations of the device depicting the distal end in varying configuration relative to the elongate shaft. FIG. 4A shows the distal end of the device in line with the elongate shaft. FIG. 4B shows the distal end of the device sitting an angle in relation to the elongate shaft. FIG. 4C shows the device's elongate shaft having a steerable section at its junction with the distal end allowing for in-situ adjustment of the angle of the distal end relative to the elongate shaft.

FIG. 5A depicts a configuration of the device and systems of the current invention wherein a gas/liquid cartridge, a control mechanism to axially slide the distal end and/or its ablation component, and an image processing component are located within the proximal end of the device (shown in a device handle) which is in turn connected to an external light source and display device.

FIG. 5B depicts a configuration of the device and systems of the current invention wherein where only the control mechanism to axially slide the distal end and/or its ablation component are housed within the proximal end of the device while other components are housed external to the proximal end (or device handle).

FIG. 6 outlines a step-by-step method to use the devices described in this invention to treat diseases via endoscopic neuroablation in the tympanic cavity.

### DETAILED DESCRIPTION OF THE INVENTION

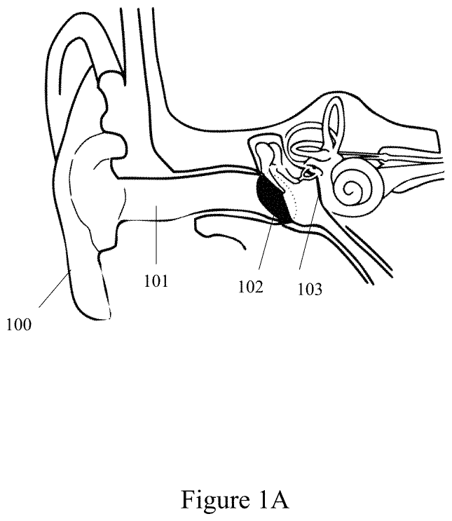

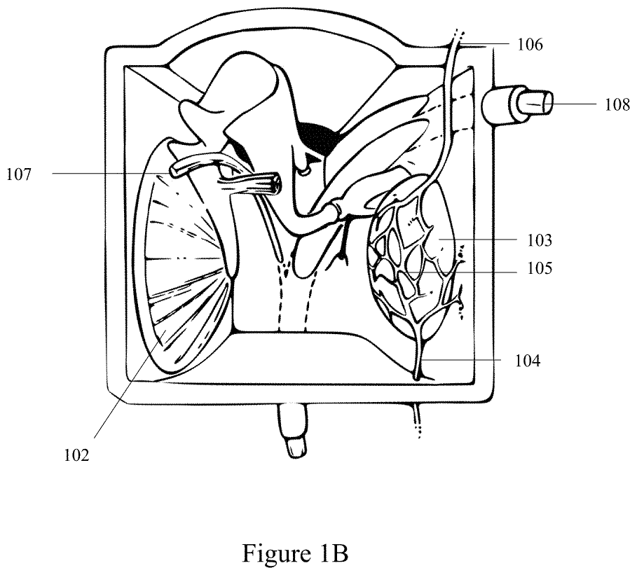

In order to provide clarity regarding the relevant anatomical structures of the ear, FIG. 1A shows the auricle 100, external auditory canal 101, tympanic membrane 102 and the promontory of the tympanic cavity 103. FIG. 1B shows a simplified close-up picture of the tympanic cavity with tympanic membrane 102, promontory 103, tympanic nerve, a branch of the glossopharyngeal nerve, 104, tympanic plexus, combination of tympanic nerve with sympathetic fibers from the internal carotid nerve, 105, lesser petrosal nerve 106 and chorda tympani 107 a branch of the facial nerve 108.

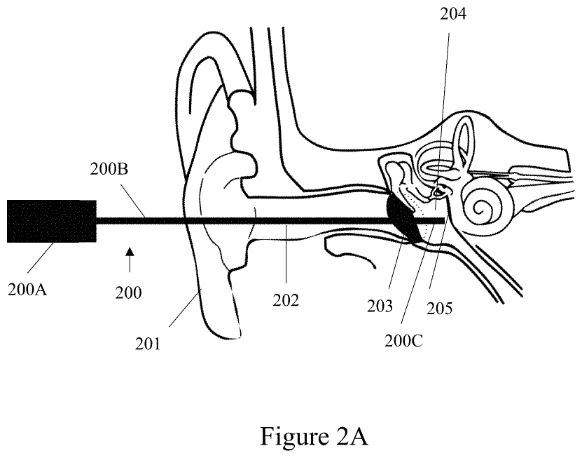

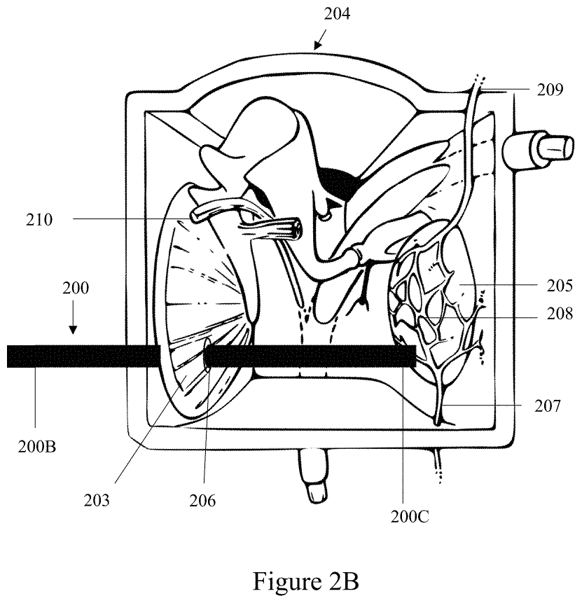

FIG. 2A shows a schematic of the device of the current invention 200 comprising proximal end 200A, elongate shaft 200B and distal end 200C. The proximal end 200A is used by an operator to navigate the distal end through the auricle 201, across the external ear canal 202, through an incision in the tympanic membrane 203 to enter tympanic cavity 204 in proximity to the promontory 205. The length of elongate shaft 200B is designed so that proximal end 200A (e.g., the device handle) remains outside of the patient's ear while distal end 200C is positioned in proximity to structures within tympanic cavity 204. The length of elongate shaft 200B is about 5-30 cm, or preferably within the range of 10 cm to 20 cm. FIG. 2B shows a close-up of the tympanic cavity 204, where the distal end 200C of device 200 has been navigated through incision 206 in the tympanic membrane 203. Incision 206 can be created using standard surgical technique and instruments prior to inserting device 200 into the patient's ear or it can be created using device 200 on its way to tympanic cavity 204. The diameter of incision 206 is about 0.5 mm to 3 mm, or preferably between 1 mm to 2.5 mm. Once inside tympanic cavity 204, distal end 200C is used to ablate targeted nerve tissues including, but not limited to the tympanic nerve 207, tympanic plexus 208, lesser petrosal nerve 209 or chorda tympani 210.

In one embodiment, in addition to ablating a nerve or nerves, device 200 is configured to illuminate and visualize the inside of tympanic cavity 204 to identify the targeted nerve or nerves and perform the procedure.

In yet another embodiment (not shown), instead of incision 206 of the tympanic membrane 203 to access the tympanic cavity 204, an incision can be made in external ear canal 202 in proximity to the tympanic membrane 203 in order to elevate a portion of tympanic membrane 203 in form of a flap to provide distal end 200C access to tympanic cavity 203.

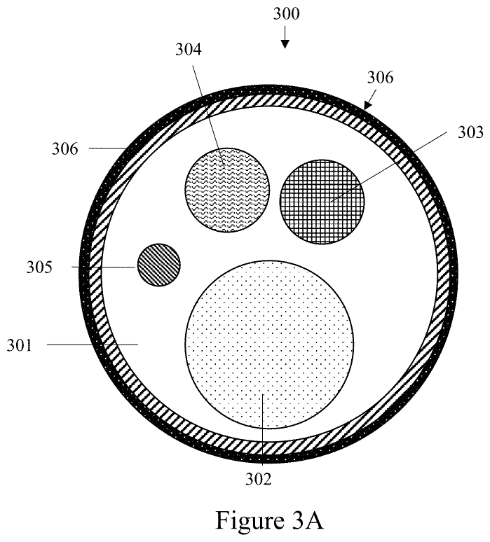

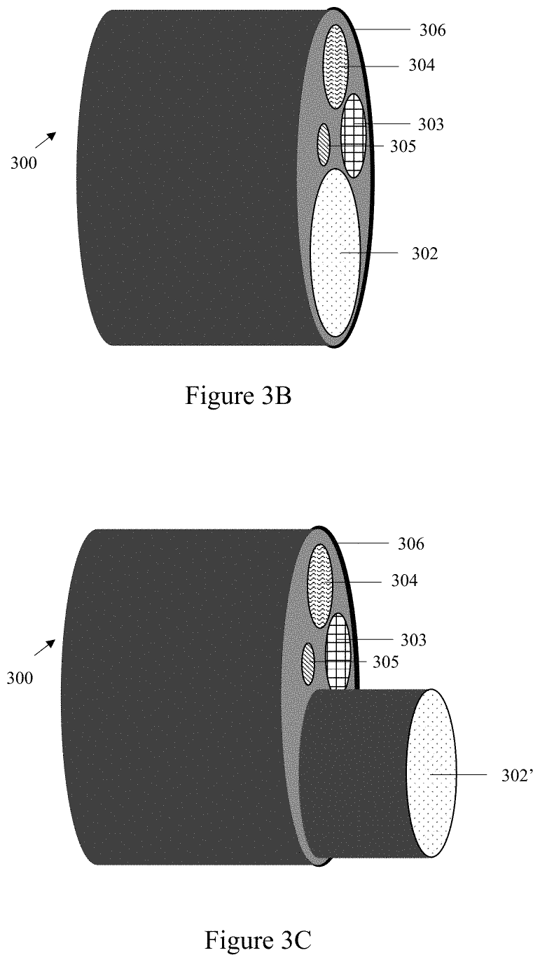

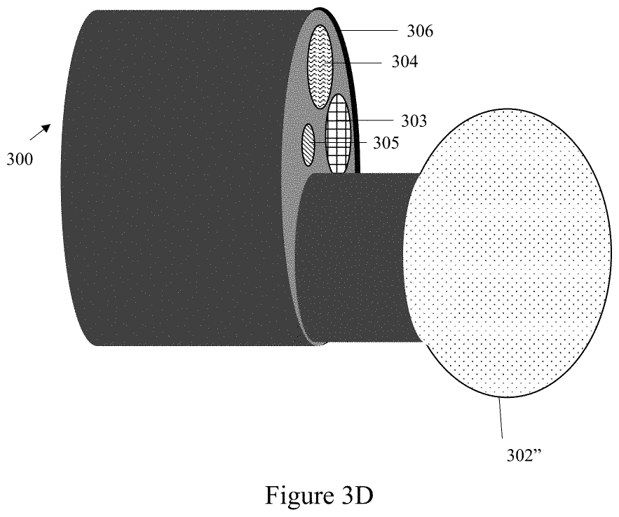

FIG. 3A shows the cross-section of distal end 300 of the device of the current invention comprising lumen 301, ablation effector 302, optional image capture component 303, optional illumination component 304, optional working channel 305, and optional cutting component 306. The diameter of distal tip 300 is such that it can navigate through a small incision in the tympanic membrane if desired and in general operate within the confines of the tympanic cavity space and is expected to be 1 mm to 3 mm or preferably between 1 mm to 2.5 mm in diameter.

Optional cutting component 306 can be used to create the desired tympanic membrane incision 202 (described previously in FIG. 2B). In one embodiment, optional cutting component 306 is designed to slide back and forth axially along all or part of the elongate shaft of the device (not shown). In another embodiment, optional cutting component 306 is designed to rotate around the perimeter of distal end 300. In yet another embodiment, optional cutting component 306 is designed to rotate around the perimeter of distal end 300 and slide along elongate shaft of the device (not shown). In one example of this configuration, initially optional cutting component 306 is positioned in the proximity of proximal shaft of the device (not shown). Once distal end 300 is navigated through the external ear canal and is positioned in the proximity of tympanic membrane (not shown), cutting component 306 is slid forward towards distal end 300 so that it comes in contact with the tympanic membrane (not shown). Cutting component 306 then rotates along the perimeter of distal end 300 while in contact with the tympanic membrane to create an incision. Once the desired incision is created, cutting component 306 is slid backward (e.g. retracted) towards the proximal end of the device (not shown) placing its cutting surface along the elongate shaft in a position that facilitates safe delivery of the device as required into the tympanic cavity.

FIG. 3B shows the side view distal end 300 comprising ablation effector 302, optional image capture component 303, optional illumination component 304, and optional working channel 305. In one embodiment of this invention, ablation effector 302 is in line and on the same plane as other components of distal end 300.

In one embodiment of the current invention ablation effector 302 is a cryoprobe using Joule's Thompson (JT) effect to create temperatures between, e.g., −20° C. to −100° C. to effect nerve ablation. In this embodiment, a compressed refrigerant gas or liquid is ported through the cryoprobe via a small internal lumen to the proximity of the tip of the cryoprobe at which point the refrigerant gas or liquid exits the internal smaller lumen to enter a coaxial larger lumen. The refrigerant gas or liquid may be contained within a reservoir located within the proximal end or externally of the proximal end and coupled to the ablation effector 302 through the elongate shaft 200B. The expansion of the refrigerant gas or liquid during transition from the smaller lumen to the larger lumen generates extremely low (cryogenic) temperatures at the tip of the cryoprobe.

In one embodiment of this invention, ablation effector 302 is a cryoprobe that uses compressed nitrous oxide gas or liquid to create the extremely low temperatures. In another embodiment, the refrigerant gas or liquid may comprise carbon dioxide as the refrigerant and in yet another embodiment, the refrigerant may comprise a chlorofluorocarbon, hydrochlorofluorocarbon, hydrofluorocarbon or any mixtures thereof to produce the extremely low temperatures.

In one embodiment, ablation effector 302 is a cryoprobe that uses liquid nitrogen to produce extreme low temperatures. In yet another embodiment, ablation effector 302 uses other methods than JT effect to produce the extremely low temperatures necessary for tissue ablation.

In another embodiment, ablation effector 302 uses heat to ablate tissue. The heat for ablation can be produced by various energy modalities including, but not limited to bipolar radiofrequency, pulsed radiofrequency, microwave or ultrasonic energy.

In one embodiment, ablation effector 302 delivers liquid or semi-liquid chemicals unto the target tissue to effect ablation. Examples of such chemicals include ethanol, phenol, or formulations or combinations thereof.

In another embodiment, ablation effector 302 delivers liquid or semi-liquid therapeutic agents to induce ablation. Examples of such agents include, but are not limited to capsaicin and its analogues (e.g. Zucapsaicin, ALGRX-4975, Nonivamide, Resiniferatoxin, or combinations thereof), sympatholytic agents such as alpha- and beta-adrenergic receptor antagonists (e.g., alpha and beta blockers), or centrally acting agents such as clonidine, guanabenz, methyldopa, minoxidil, and reserpine.

In one embodiment, the diameter of ablation effector 302 is about 0.5 mm to 3 mm. In a preferred embodiment, the diameter of ablation effector 302 is between 0.5 mm to 2 mm.

In one embodiment, optional image capture 303 is a miniature or subminiature CMOS and optional illumination component 304 is fiber optic bundle connected to an external light source (not shown). In another embodiment, optional illumination component 304 is a light emitting diode (LED) or alternatively a laser light source. The CMOS would be positioned proximal to a glass or plastic lens (also not shown) used to provide the desired focal length and field of view within the tympanic cavity.

FIG. 3C shows the side view of distal end 300 comprising ablation effector 302′, optional image capture component 303, optional illumination component 304, and optional working channel 305, where ablation effector 302′ is positioned distal (slide forward axially) relative to the other components shown at distal end 300. This configuration allows for better visualization of the ablation procedure occurring at the tip of ablation effector 302′ by positioning it in the field of view (e.g., in front of or distal to) the image capture component 303.

In one embodiment, ablation effector 302′ is positioned about 1 mm to 25 mm distal to other components of distal end 300. In another embodiment, the distance between the most distal tip of ablation effector 302′ relative to the other components of distal end 300 is between 1 mm to 10 mm or even more preferably between 1 mm to 5 mm.

In one embodiment, the ablation effector 302′ is designed to slide axially back and forth relative to other components of distal end 300. During navigation to the tympanic cavity, ablation effector 302′ is in line with other components of distal end 300. Once within the tympanic cavity and after the nerve tissue targeted for ablation is visually identified, ablation effector 302 (as depicted in FIG. 3B) is slid forward (distally) relative to other components of distal end 300 to the position of ablation effector 302′ (as depicted in FIG. 3C) placing the distal most tip of the ablation effector 302 in front of or distally and within the field of view and focal length of image capture component 303. Upon completion of the procedure and prior to navigating out of the tympanic cavity, ablation effector 302′ is slid backward or retracted axially to position of ablation effector 302 (as depicted in FIG. 3B) in line with other components of distal end 300 facilitating navigation of the device out of the patient's ear.

FIG. 3D shows a side view of distal end 300 comprising ablation effector 302″, image capture component 303, illumination component 304, and optional working channel 305. In this configuration, ablation effector 302″ has an expandable tip. During navigation to the target tissue, ablation effector 302″ is in a collapsed state to facilitate navigation (similar to ablation effector 302′ depicted in FIG. 3C). Once ablation effector 302″ is in proximity to the targeted tissue or nerve(s), it can change form a collapsed state to an expanded state to facilitate ablation of larger surface area of tissue possibly also improving conformity of ablation effector 302″ to uneven tissue surface morphologies. Upon completion of the ablation procedure and prior to navigation (e.g., retraction) out of a patient's ear, ablation effector 302″ is returned to the collapsed state (similar to ablation effector 302′ as depicted in FIG. 3C) to ease retraction from the anatomy and to reduce unintended trauma or tissue damage during retraction.

It is contemplated that ablation effector 302″ can also slide axially relative to other components of distal end 300 in addition to assuming collapsed and expanded states. For example, during navigation to the target tissue, ablation effector 302″ can be arranged in line with other components of distal end 300 (in a configuration similar to ablation effector 302 depicted in FIG. 3B). Once effector 302″ is in proximity of targeted tissue, it can be slid forward (e.g., distally or axially) relative to other components of distal end 300 (in a configuration similar to ablation effector 302′ depicted in FIG. 3C) and change from collapsed state to expanded state. Upon completion of the ablation procedure, ablation effector 302″ can then revert back to its original collapsed state and slide backward axially to line it up with other components of distal end 300 prior to navigation (e.g., retraction) out of the patient's ear.

In one embodiment the ablation effector 302″ is a cryoprobe using the Joule's Thompson (JT) effect to create temperatures between −20° C. to −100° C. to effect ablation and the expandable portion of ablation effector 302″ is a balloon. In this configuration the refrigerant gas used to cause JT effect is also used to simultaneously expand the balloon. Upon the completion of the ablation procedure, the flow of the refrigerant gas is stopped bringing the temperature of ablation effector 302″ back to environmental temperatures at the same time causing deflation of the balloon.

In another embodiment, the expandable portion of ablation effector 302″ is a structure can be fabricated from thermally conductive materials capable of assuming a collapsed and an expanded state. In one embodiment, the thermally conductive material is a metal (e.g. Nitinol) which can be expanded and collapsed based on the shape-memory properties of the material.

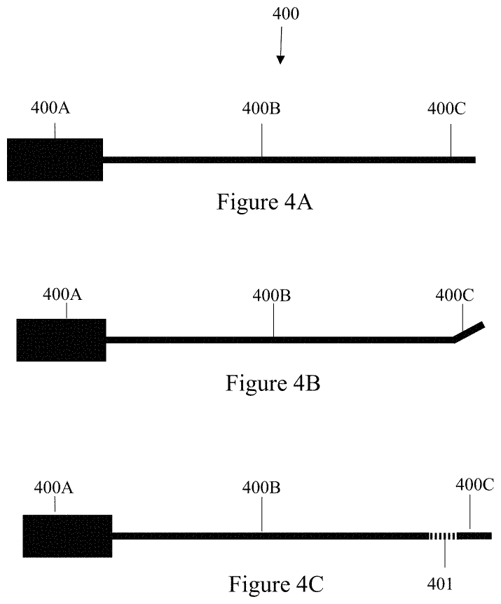

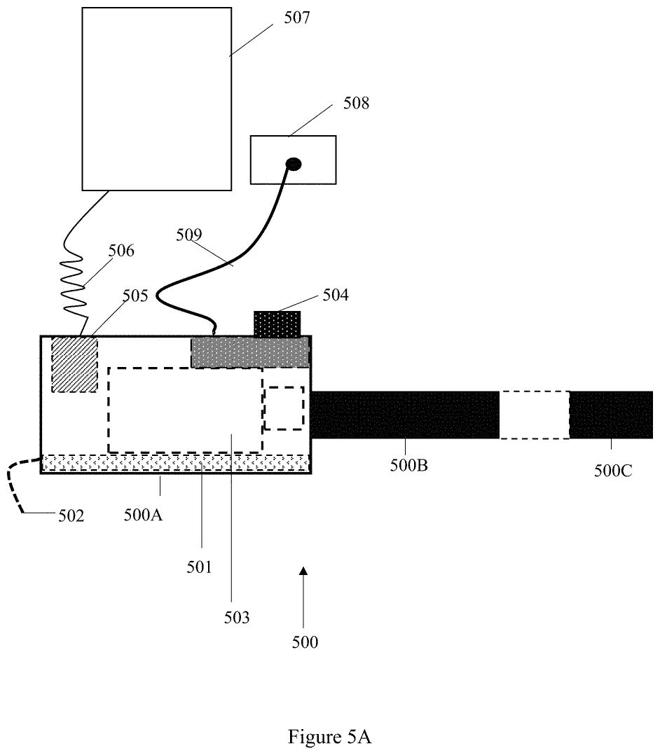

FIG. 4A depicts the schematic of device 400 of this invention with proximal end 400A, elongate shaft 400B and distal tip 400C. In this embodiment distal tip 400C is in line (on axis with) and in a straight configuration relative to elongate shaft 400B. FIG. 4B depicts device 400 where distal end 400C sits at an angle relative to elongate shaft 400B. It is contemplated that the angle between distal end 400C and elongate shaft 400B is between, e.g., 0 degrees to 120 degrees. In a preferred embodiment, this angle is between, e.g., 0 degrees to 90 degrees. The contemplated angle is designed to facilitate and ease access to nerve tissues situated at various angles relative to the entry point (e.g., the incision point in the tympanic membrane) of device 400 into tympanic cavity.

In one embodiment of the current invention, distal tip 400C includes a specific bend angle at distal end 400C. In another embodiment, elongate shaft 400B is composed of a malleable material or construction that allows intraprocedural adjustment or tailoring or bending of distal end 400C by the operator in accordance with the specific geometric or anatomic requirements of a specific patient's ear.

FIG. 4C depicts a schematic of device 400 wherein elongate shaft 400B has a steerable portion 401, the movement of which can be controlled by the operator. In this embodiment, the bend angle between distal end 400C relative to elongate shaft 400B can be adjusted in-situ (intraprocedurally) as desired by the operator.

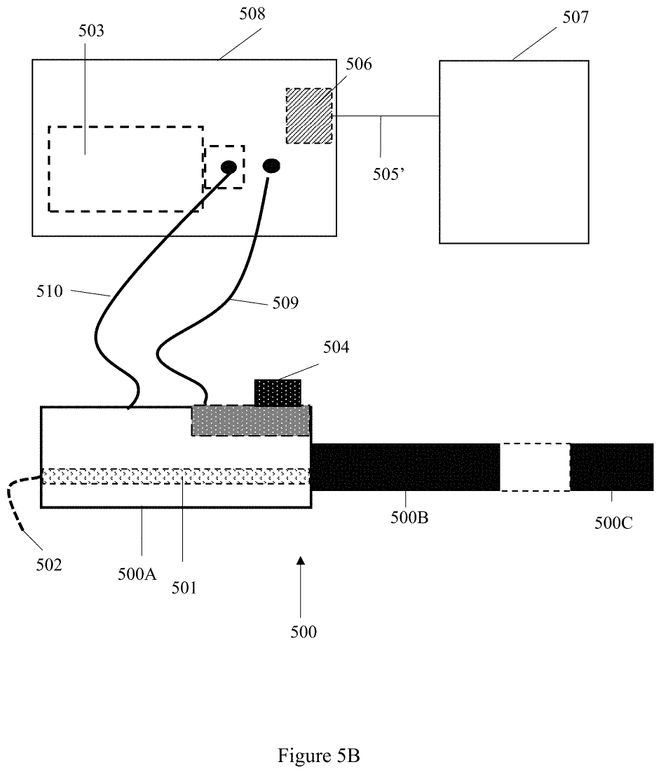

FIG. 5A depicts the schematic of device 500 comprising proximal end 500A, elongate shaft 500B and distal end 500C, where distal end 500C comprises a cryoprobe as ablation effector (not shown) and a working channel component (not shown). Proximal end 500A houses lumen 501 that can optionally be connected to a vacuum source using tubing 502 or can be used to pass surgical tools from proximal end 500A through elongate shaft 500B to the surgical site. Lumen 501 is connected to the working channel of the device (not shown). Proximal end 500A also houses compressed liquid or gas refrigerant cartridge 503 which is in fluid communication with distal tip 500C. Control mechanism 504 is also placed within proximal end 500A. Control mechanism 504 allows the operator to control the movement of the ablation effector, adjust the bend angle of distal tip 500C relative to elongate shaft 500B, and/or control the movements of the cutting component of distal end 500C (not shown). Image processing component 505 is in wireless communication with the image capture device of distal end 500C (not shown) and in electronic communication with image display device 507 through cable 506. Proximal end 500A is connected to external light source 508 through cable 509. Cable 509 is in communication with the illumination component of distal end 500C (not shown).

As middle ear surgery involves operating in small spaces and around delicate/sensitive anatomical structures, and in order to provide the operator with better control and more precise manipulation of the device of the current invention, it would be preferrable to reduce the weight and volume of the proximal end 500A of the device. FIG. 5B depicts schematics of device 500 with proximal end 500A, elongate shaft 500B and distal end 500C. Proximal end 500A houses lumen 500A that can be optionally connected to a vacuum source through tube 502 or be used as a general-purpose working channel. It also houses control mechanism 504 that can control the movement and operation of the ablation effector (not shown), distal end 500C in relation to elongate shaft 500B, and/or the cutting component (not shown). Compressed liquid or gas refrigerant cartridge 503 and image processing component 506 are housed within external structure 508 along with the illumination light source. Cartridge 503 is in liquid communication with proximal end 500A and distal end 500C through tube 510. Image processing component 506 can be in wireless communication with image capture device component of distal end 500C (not shown) and electronically connected to image display device 507 using cable 505′in the form of, e.g., an HDMI or USB connector or the like well-known in the art. Proximal end 500A is connected to external light source 508 through cable 509. Cable 509 is in communication with the illumination component of distal end 500C (not shown).

It is contemplated herein that portions of the various previously described embodiments may be rendered disposable thereby eliminating or reducing the cost and burden of instrument cleaning, reprocessing or resterilization where practicable.

Possible configurations of components are not limited to embodiments described herein. Other configurations to impart practicality and/or ease of use to the device are contemplated by the authors.

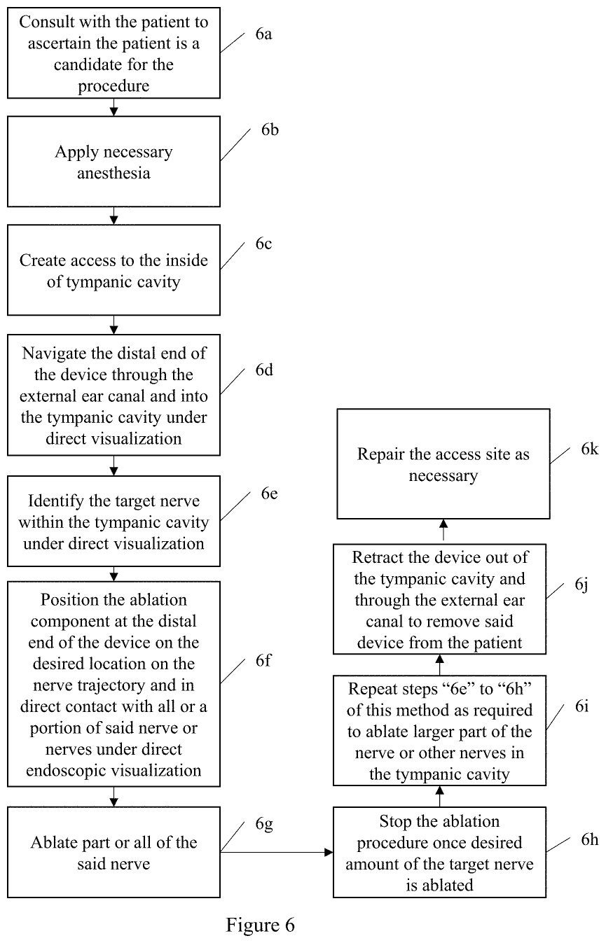

Step by step methods of using devices of current invention to treat diseases related to nerves innervating or passing through tympanic cavity are depicted in FIG. 6 (steps 6a to 6k) and are as follows:
a. Consult with the patient and ascertain that the patient is a candidate for the current procedure,
b. Apply necessary anesthesia,
c. Create access to the inside of the tympanic cavity,
d. Navigate the distal end of the device through the external ear canal and into the tympanic cavity under direct visualization,
e. Identify the target nerve or nerves within the tympanic cavity under direct visualization,
f. Position the ablation component at the distal end of the device on the desired location on the nerve trajectory and in direct contact with all or a portion of said nerve or nerves under direct direct visualization,
g. Ablate all or a portion of said nerve or nerves,
h. Stop the ablation procedure once the desired amount of ablation of the target nerve or nerves is completed,
i. Repeat steps “f” to “i” of this method as required to further ablate the same or different nerve or nerves in the tympanic cavity,
j. Retract the device out of the tympanic cavity and through the external ear canal to remove said device from the patient.
k. Repair the access site as necessary.
   While individual steps are illustrated and described in FIG. 6, some of the steps may be optionally omitted in other embodiments and/or taken in a different order as desired or necessary. Furthermore, any combination of device features described may be used in any of the methods described in any number of combinations.

Prior to the ablation procedure the physician might conduct a diagnostic procedure to ensure of the suitability of the ablation target. In some embodiments such diagnostic step might include injecting or applying an anesthetic to the target location.

Direct visualization includes but not limited to using an endoscopes, rigid or flexible, or a microscope.

In one embodiment of the method, the device includes or directly incorporates the visualization and/or illumination components. In another embodiment separate visualization and/or illumination instruments or tools are used.

In one embodiment of the method, in order to access the tympanic cavity, an incision is made on the tympanic membrane. In another embodiment, an incision is made in the external ear canal in the proximity of the tympanic membrane to elevate a tympanomeatal flap to provide access to the tympanic cavity. In another embodiment facial recess approach is used to access the tympanic cavity.

In one embodiment of the methods, the device includes a cutting component that can be used to create access to tympanic cavity, thereby combining the elements of steps c and d.

In one embodiment of the method described herein, the step to identify the target nerve or nerves can include using a nerve tissue visualization aid (e.g. dyes) or other techniques to distinguish nerve tissue from surrounding tissues. In another embodiment, the step to identify target nerve includes methods of nerve stimulation or nerve monitoring commonly known in the art.

In one embodiment of the method contemplated in this invention, the ablation effector component of the device is a cryoprobe that can provide temperatures in the range of, e.g., −20° C. to −100° C. to effect cryoablation of the main branch of the tympanic nerve and/or the tympanic plexus located on or near the promontory of the tympanic cavity of a patient. In this example, the method and devices described herein are used to treat dysfunctions and/or diseases including, but not limited to referred otalgia (e.g. otalgia from TMJ disorders), otic neuralgia, glossopharyngeal neuralgia, headache such as migraine, parotid gland fistula, parotitis, drooling, autonomic dysfunction and/or the Frey syndrome.

In another embodiment of the method contemplated in this invention, the ablation effector component of the device is a cryoprobe that can provide temperatures in the range of −20° C. to −100° C. degrees to effect cryoablation of the chorda tympani nerve located in the tympanic cavity of a patient. In this example, the method and devices described herein are used to treat dysfunctions and/or diseases including, but not limited to taste disturbances, such as autonomic dysfunction, hypogeusia, ageusia or dysgeusia, excessive drooling, retronasal olfaction, or orosensory hyperactivity such as burning mouth syndrome.

In another embodiment of the method contemplated in this invention, the ablation component of the device is a cryoprobe that uses temperatures in the range of −20° C. to −100° C. degrees to effect cryoablation of both the chorda tympani nerve and the tympanic nerve located in the tympanic cavity of a patient for example to treat obesity.

In another embodiment of the method contemplated in this invention, the ablation component of the device is a cryoprobe that uses temperatures in the range of −20° C. to −100° C. degrees to effect cryoablation of Arnold's nerve.

After the cryoablation of the target nerve, the patient will lose the function of that nerve for a period of time, after which the nerve is expected to regenerate into healthy tissue or nerve tissue exhibiting substantially less dysfunction than prior to the ablation procedure. In some patients, it might be necessary to repeat the procedure to improve efficacy or to extend the duration of the ablative (therapeutic) effects on the targeted nerve or nerves.

Other medical therapies such as use of pharmaceutical agents can be conducted in combination with the methods described herein in order to improve the clinical outcomes of the patient.

What is claimed is:

1. A method for treating dysfunctions or diseases in a patient, comprising:
creating access to a tympanic cavity of a patient;
introducing a treatment device into the tympanic cavity of the patient, the treatment device having a proximal end, a distal end, an elongate shaft therebetween, and an ablation effector disposed at or near the distal end of the device;
advancing the distal end of the device under direct visualization into proximity of a nerve or nerves within the tympanic cavity; and
ablating the nerve or nerves using the ablation effector to reduce at least one symptom of nerve dysfunction or disease.

2. The method of claim 1 wherein ablating the nerve or nerves comprises ablating a tympanic nerve.

3. The method of claim 1 wherein ablating the nerve or nerves comprises ablating a tympanic plexus.

4. The method of claim 1 wherein ablating the nerve or nerves comprises ablating a chorda tympani.

5. The method of claim 1 wherein ablating the nerve or nerves comprises ablating a lesser petrosal nerve.

6. The method of claim 1 wherein ablating the nerve or nerves comprises ablating a deep petrosal nerve.

7. The method of claim 1 wherein ablating the nerve or nerves comprises ablating a nervous intermedious.

8. The method of claim 1 wherein ablating the nerve or nerves comprises ablating a communicating branch of a facial nerve to a tympanic plexus.

9. The method of claim 1 wherein ablating the nerve or nerves comprises ablating Arnold's nerve.

10. The method of claim 1 wherein ablating the nerve or nerves comprises ablating a portion or branch of a facial nerve or a greater petrosal nerve.

11. The method of claim 1 wherein ablating the nerve or nerves comprises ablating a portion of caroticotympanic nerves.

12. The method of claim 1 wherein the dysfunctions or diseases to be treated is selected from the group consisting of otitis media, otitis media with effusion, chronic otitis media with effusion, chronic suppurative otitis media, referred otalgia, otalgia from a TMJ disorder, otic neuralgia, fistula of parotid gland, parotitis, Frey syndrome, autonomic dysfunction, disturbances of sense of tase including hypogeusia, ageusia or dysgeusia, excessive drooling, headache, retronasal olfaction, orosensory hyperactivity, and obesity.

13. The method of claim 1 wherein creating access to the tympanic cavity comprises making an incision on a tympanic membrane.

14. The method of claim 13 wherein a diameter of the incision on the tympanic membrane does not exceed 3 mm.

15. The method of claim 1 wherein the treatment device includes a cutting component to create an incision on a tympanic membrane.

16. The method of claim 1 wherein creating access to the tympanic cavity comprises making an incision in an external ear canal and elevating a tympanomeatal flap.

17. The method of claim 1 wherein creating access to the tympanic cavity comprises following a facial recess approach.

18. The method of claim 1 wherein the distal end of the treatment device includes a visualization or image capture component.

19. The method of claim 18 wherein the visualization or image capture component of the device is a CMOS camera.

20. The method of claim 18 wherein the ablation effector of the distal end of the treatment device is configured to be positioned in a field of view of the visualization or image capture component.

21. The method of claim 18 wherein the distal end of the device includes an illumination component.

22. The method of claim 21 wherein the ablation effector of the distal end of the treatment device is configured to slide axially relative to the visualization or image capture component and the illumination component.

23. The method of claim 18 wherein the treatment device includes a heating or cooling component to protect the visualization or image capture component from temperature changes caused by the ablation effector.

24. The method of claim 1 wherein advancing the distal end of the device comprises reconfiguring an expandable structure to transition from a collapsed configuration to an expanded configuration.

25. The method of claim 1 wherein ablating the nerve or nerves comprises ablating the nerve or nerves via cryoablation.

26. The method of claim 1 wherein ablating the nerve or nerves comprises ablating via heat using bipolar radiofrequency, pulsed radiofrequency, microwave, or ultrasonic energy.

27. The method of claim 1 wherein ablating the nerve or nerves comprises ablating the nerve or nerves via ethanol and/or phenol.

28. The method of claim 1 wherein ablating the nerve or nerves comprises ablating the nerve or nerves via a therapeutic agent.

29. The method of claim 28 wherein the therapeutics agent includes capsaicin and its analogues, sympatholytic agents, and/or centrally acting agents.

30. The method of claim 1 wherein ablating the nerve or nerves comprises ablating via a cryogenic ablation agent delivered through the distal end of the treatment device.
